(19) United States
(12) Patent Application Publication
(10) Pub. No.: US 2004/0265276 A1
(43) Pub. Date: Dec. 30, 2004

(54) Medicinal combination useful for in vivo exogenic transfection and expression

(76) Inventors: Michel Perricaudet, Ecrosnes (FR); Lucienne Chatenoud, Paris (FR); Hedi Haddada, Bg la Reive (FR); Jean-Francois Bach, Paris (FR); Michelle Webb, London (GB)

Correspondence Address: WILEY, REIN & FIELDING, LLP, ATTN: PATENT ADMINISTRATION, 1776 K. STREET N.W., WASHINGTON, DC, 20006, US

(21) Appl. No.: 10/823,682

(22) Filed: Apr. 14, 2004

Related U.S. Application Data
(63) Continuation of application No. 08/894,246, filed on May 22, 1998, now abandoned.
Application No. 08/894,246 is a 371 of international application No. PCT/FR96/00218, filed on Feb. 12, 1996.

(30) Foreign Application Priority Data
Feb. 14, 1995 (FR) 95/01662

(51) Int. Cl. A61K 48/00; A61K 39/395

(52) U.S. Cl. 424/93.2; 424/131.1

(57) ABSTRACT

A medical combination of at least one immunosuppressive agent and at least one recombinant adenovirus with a genome that includes a first recombinant DNA containing a therapeutic gene, and a second recombinant DNA containing an immunoprotective gene, for consecutive, intermittent and/or simultaneous use in in vivo and/or ex vivo exogenic transfections.

The present invention relates to the field of gene therapy and in particular to the use of adenovirus for expressing a therapeutic gene of interest. It relates, more specifically, to a novel method for treating pathologies of genetic origin, which method is based on the combined use of two types of therapeutic agents.

Gene therapy consists in correcting a deficiency or an anomaly (mutation, aberrant expression, etc.) by introducing genetic information into the affected cell or organ. This genetic information can be introduced either in vitro or ex vivo into a cell which has been removed from the organ, with the modified cell then being reintroduced into the organism, or else directly in vivo into the appropriate tissue. In this second case, a variety of different physical techniques exist for transfection, including the use of viruses as vectors. In this respect, a variety of different viruses have been tested for their ability to infect particular cell populations. These viruses include, in particular, retroviruses (RSV, HMS, MMS, etc.), the HSV virus, adeno-associated viruses and adenoviruses.

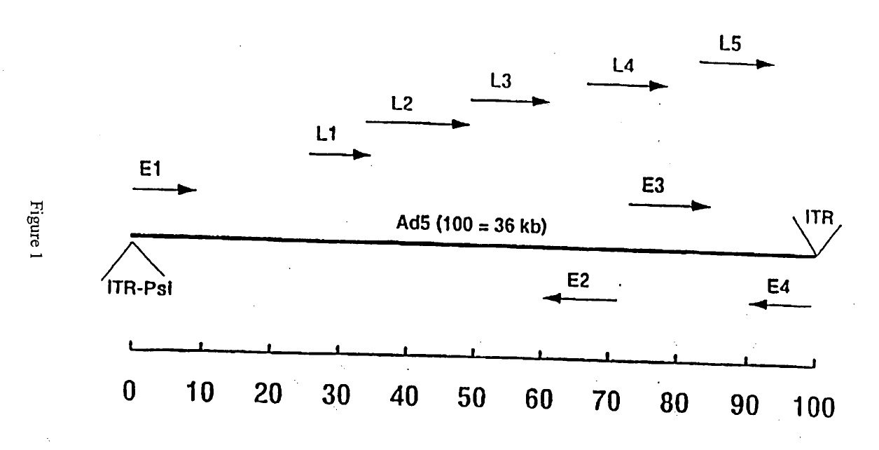

Among these viruses, the adenoviruses exhibit some properties which are favourable in relation to use in gene therapy. They have a rather broad host spectrum, are capable of infecting quiescent cells and do not integrate into the genome of the infected cell. The adenoviruses are viruses which contain linear double-stranded DNA of about 36 kb in size. Their genome encompasses, in particular, an inverted repeat sequence (ITR) at their end, an encapsidation sequence, early genes and late genes (cf. FIG. 1). The principal early genes are the genes E1 (E1a and E1b), E2, E3 and E4. The principal late genes are the genes L1 to L5.

In view of the adenovirus properties mentioned above, these viruses have already been used for m transferring genes in vivo. To this end, different adenovirus-derived vectors have been prepared which incorporate a variety of different genes (β-gal, OTC, α-IAT, cytokines, etc.). In each of these constructs, the adenovirus was modified in such a way as to render it incapable of replicating in the infected cell. Thus, the constructs which are described in the prior art are adenoviruses from which the E1 (E1a and/or E1b) and, possibly, E3 regions have been deleted, with a hetero-logous DNA sequence being inserted in their stead (Levrero et al., Gene 101 (1991) 195; Gosh-Choudhury et al., Gene 50 (1986) 161).

However, as in the case for all known viruses, administration of a wild-type virus (Routes et al., J. Virol. 65 (1991) 1450) or of a recombinant virus which is defective for replication (Yang et al., PNAB (1994) 4407) induces a substantial immune response.

The primary aim of the immune system is the integrity of the individual or the integrity of “self”. It leads to the elimination of infectious agents and the rejection of transplants and tumours without, however, these powerful defence mechanisms of the organism turning against it and giving rise to autoimmune diseases. This state of non-response with regard to “self” antigens when foreign antigens are eliminated is defined as a state of physiological tolerance. In order to eliminate foreign agents, the immune system develops two types of mechanisms. The first is the production of specific anti-bodies by the B lymphocytes; this is termed humoral immunity. These antibodies fix the antigen and either inactivate it or eliminate it from the organism. The second defence mechanism involves cellularimmunity and employs T lymphocytes, among these the cytotoxic T lymphocytes which carry a specific receptor for the antigen in question. Recognition of the antigen by the T receptor necessitates the latter being expressed in association with proteins which are encoded via the genes of the major histocompatibility complex or class I and class II MHC.

Consequently, this immune response, which is developed against the infected cells, constitutes a major obstacle to the use of viral vectors in gene therapy since (i) by inducing destruction of the infected cells it limits the period during which the therapeutic gene is expressed and hence the therapeutic effect, (ii) it induces, in parallel, a substantial inflammatory response, and (iii) it brings about rapid elimination of the infected cells after repeated injections. It will be understood that the amplitude of this immune response against infected cells varies according to the nature of the organ which sustains the injection and according to the method of injection which is employed. Thus, expression of the β-galactosidase encoded by a recombinant adenovirus which is administered into the muscle of immunocompetant mice is reduced to minimum levels 40 days after the injection (Kass-Eisler et al., PNAS 90 (1993) 11498). In the same way, the expression of genes which have been transfected into the liver using adenoviruses is significantly reduced in the 10 days following the injection (Yang Y et al. 1994 immunity 1 433-442) and expression of factor IX which was transferred using adenovirus into the hepatocytes of hemophiliac dogs disappeared at 100 days after the injection (Kay et al. PNAS 91 (1994) 2353).

From the point of view of exploiting vectors derived from adenoviruses for the purpose of gene therapy, it therefore seam necessary to control the immune response which is developed against them or against the cells which they are infecting.

From the above, it follows that activation of the immune system first of all requires recognition by the system of elements which are foreign to the organism (non-self or modified self) such as vectors derived from adenoviruses, which would normally be destroyed. In recent years, immunointervention strategies have been developed whose aim is to create a “permissive” immune environment, that is to say induce a state of tolerance with regard to predefined foreign antigens.

It is precisely at this level that the present invention intervenes. The invention is directed towards preventing the rapid elimination of the adenoviruses from the infected cells and hence towards prolonging, in a consistent manner, the in vivo expression of the therapeutic gene which they are carrying.

Recently, the Applicant has demonstrated that the co-expression of certain genes in the infected cells is able to induce an immunoprotective effect and thus enable the vectors and/or the infected cells to evade the immune system. The Applicant has, in particular, developed adenoviruses in which expression of a gene of therapeutic importance is coupled to that of an immunoprotective gone (FR No. 9412346). This gene can, in particular, be a gene whose product acts on the activity of the major histocompatibility complex (NEC) or on the activity of the cytokinen, thereby making it possible to reduce considerably, if not suppress, any immune reaction against the vector or the infected cells. These gene products at least partially inhibit expression of the MHC proteins or presentation of the antigen, advantageously resulting in a significant reduction of the immune reaction against the vector or the infected cells, and hence a prolonged therapeutic effect.

Unexpectedly, the Applicant demonstrated that it was possible significantly to prolong, over time, the therapeutic effect of such a vector by associating it with an immunosuppressant. Elimination of the vector in question and/or destruction of the infected cells, by the immune system, is/are found to be retarded over time by a period which-in markedly greater than that which might have been expected by the simple juxtaposition of the immunoprotective effects of the said vector and the immunosuppressant. Advantageously, the medicinal combination, which in a subject of the present invention, induces a phenomenon of pseudo-inertial of the immune system, which phenomenon favours expression in the long term of a therapeutic gene.

Within the meaning of the invention, immunosuppressant indicates any compound which is able to inhibit, wholly or in part, at least one immune signalling pathway. In general, immunosuppressants are routinely used in transplantation, with the aim of preventing allograft rejection, and in the treatment of certain autoimmune diseases. The products which are customarily used are either chemical immunosuppressants such as corticosteroids, azathioprins, cyclosporin or FK506, or biological immunosuppressants such as polyclonal or monoclonal antibodies. The first category of immunosuppressants, and among these cyclosporin and FK506, in particular, exert a substantial inhibitory effect on the production of cytokines, such as interleukin 2, which play an essential role in the differentiation and proliferation of the lymphocytic cells. Unfortunately, for this type of immunosuppressant to be effective, they have to be administered continuously, something which sooner or later runs into the problem of their toxicity. Thus, azzathioprine is potentially myelotoxic while cyclosporin is nephrotoxic and can also bring about hypertension or neurological disorders.

As regards the antibodies, more particularly, these are antibodies which are directed against the lymphoid cells of the immune system. The first antibody which was used as an immunosuppressant is anti-CD3, which is directed against the T lymphocytes. Its target is the one of the polypeptidechains of the CD3 molecule which forms the receptor for the T cell antigen. There then follows a functional inactivation of the CD3+ T cells which are recognized by the antibody. As regards the problem which is of interest in the present came, administration of an immunosuppressant of this type together with that of a recombinant adenovirus containing a therapeutic gene would be in a position to block the immune reaction of the host with regard to the viral vector and/or its products which are expressed on the surface of the infected cells. Anti-CD4, -CD2, -CD8, -CD28, -B7, -ICAM-1 and -LFA-1 antibodies can be used on the same principle.

The Applicants have has now developed a novel method of treatment which is particularly efficient in substantially delaying, if not inhibiting, the reaction of the immune system without raising any toxicity problem.

More specifically, the present invention ensues from the demonstration of a particularly substantial synergistic effect which is associated with the combined use of a recombinant adenovirus, in which expression of a gene of therapeutic importance is coupled to that of an immunoprotective gene, such as previously described, and of at least one immunosuppressive agent.

The present invention therefore relates, initially, to a medicinal combination of at least one immunosuppressive agent and at least one recombinant adenovirus whose genome comprises a first recombinant DNA containing a therapeutic gene and a second recombinant DNA containing an immunoprotective gene, for consecutive, intermittent and/or simultaneous use over time, which can be used for exogenous transfections in vivo and/or ex vivo.

As indicated above, the invention is based, in particular, on the demonstration of a synergistic effect between the activity of the immunosuppressive agent and the effect of the expressed immunoprotective gene on the expression of the therapeutic gene.

This combined use makes it possible to achieve a therapeutic effect which is markedly prolonged and advantageously requires doses which are significantly reduced, in particular as regards their content of immuosuppressive agent.

As indicated further below, the two components of the combined treatment of the present invention can be used consecutively, intermittently and/or simultaneously over time. Preferably, the immunosuppressive agent is injected before and after injection of the adenovirus. According to this method of implementing the present m invention, the administration of the immunosuppressant can be spaced out over time and, more preferably, be repeated regularly. In this particular cage, the two components are packaged separately. When administration takes place simultaneously, they can be mixed as required before being administered together or, on the other hand, they can be administered simultaneously but separately. In particular, the routes by which the two agents are administered can be different.

According to the present invention, any compound which is able to inhibit, wholly or in part, at least one immune signalling pathway can be used as the immunosuppressive agent. The compound can be selected, in particular, from cyclosporin, FK506, azathioprine, corticosteroids and any monoclonal or polyclonal antibody. Use in preferably made of antibodies which are able to inactivate immune molecules or induce destruction of the immune cells carrying these molecules. Anti-CD4, -CD3, -CD2, -CD8, -CD28, -B7, -ICAM-1 and -LPA-1 antibodies can, in particular, be used as antibodies. Use can also be made of hybrid molecules such as CTLA4Ig, a protein fusion between the CTLA-4 molecule (a homologue of CD28) and an immunoglobulin. The GlFc site of this molecule is found to be able to inhibit activation of the T cells by binding to the B7 molecule (D. J; Lenschow; Science, 257, 789, 1992). It is obvious that the scope of the present invention in in no way limited to the immunosuppressants enumerated above. These immunosuppressants can be employed in isolation or in combination.

The recombinant DNAs which are present in the genome of the adenovirus which is employed in accordance with the present invention are DNA fragments which contain the gene (therapeutic or immunoprotective) under consideration and, where appropriate, signals which enable it to be expressed, and which are constructed in vitro and then inserted into the genome of the adenovirus. The recombinant DXAs which are used within the scope of the present invention can be complementary DNAe (cDNAs), genomic DNAs (gDNAs), or hybrid constructs which consist, for example, of a cDXK in which one or more introns is/are inserted. They can also be synthetic or semisynthetic sequences. These DHAs can be of human, animal, vegetable, bacterial, viral, etc. origin. Use is particularly advantageously made of cDNAs or of gDNAs.

Any gene which encodes a product having a therapeutic effect may be mentioned as a therapeutic gone which can be used for constructing the vectors of the present invention. The product which is thus encoded can be a protein, a peptide, an RNA, etc.

A protein product can be homologous with regard to the target cell (that is, it can be a product which is normally expressed within the target cell when the latter is not exhibiting any pathology). In this case, expression of a protein makes it possible, for example, to compensate for insufficient expression in the cell or for expression of a protein which is inactive or weakly active due to a modification, or even to overexpress said protein. The therapeutic gene can also encode a mutant of a cell protein, which mutant has an increased stability, a modified activity, etc. The protein product can also be heterologous with regard to the target cell. In this case, an expressed protein can, for example, supplement or provide an activity which is deficient in the cell, thereby permitting the latter to resist a pathology, or else stimulate an immune response.

Those therapeutic protein products within the meaning of the present invention which may more specifically be mentioned are enzymes, blood derivatives, hormones, interleukins, interferons, TNF, etc. (FR 9203120), growth factors, neurotransmitters or their precursors or enzymes for synthesizing them, trophic factors: BDNF, CNTF, NGF, IGF, GMF, aFGF, bFGF, NT3, NT5, HARP/pleiotrophin, etc.; apolipoproteins: ApoAI, ApoAIV, ApoE, etc. (FR 93 05125), dystrophin or a minidystrophin (FR 9111947), the CFTR protein associated with mucoviscidosis, tumour-suppressing genes: p53, Rb, Rap1A, DCC, k-rev, etc. (FR 93 04745), genes encoding for factors involved in coagulation: factors VII, VIII and IX, genes intervening in DNA repair, etc.

As indicated above, the therapeutic gene can also be an antisense gene or sequence whose expression in the target cell makes it possible to control the expression of genes or the transcription of cellular mRNAs. Such sequences can, for example, be transcribed in the target cell into RHAs which are complementary to cellular mgs and thereby block translation of the latter into protein, in accordance with the technique described in Patent EP 140 308. Antisense sequences also include sequences encoding ribozymes, which are able selectively to destroy target RNAs (EP 321201).

The therapeutic genes can be of human, animal, vegetable, bacterial, viral, etc. origin. They can be obtained by any technique known to the person skilled in the art and, in particular, by screening libraries, by chemical synthesis or else by mixed methods including chemical or enzymic modification of sequences obtained by screening libraries.

The immunoprotective gene which in used within the scope of the present invention can be of different types. As previously explained, it is a gene whose product acts on the activity of the major histocompatibility complex (MHC) or on the activity of the cytokines. It is preferably a gene whose product at least partially inhibits expression of the MHC proteins or antigen presentation. AB preferred examples, mention may be made of certain genes contained in the adenovirus E3 region, the herpes virus gone ICP47 or the cytomegalovirus gene UL18.

The E3 region of the adenovirus genome contains different reading frames which, by means of alternative splicing, give rise to different proteins. Among these, the Gp19k (or E3-19k) protein is a glycoaylated transmembrane protein which is located in the membrane of the endoplasmic reticulum (RE). This protein encompasses a luminal domain which binds MHC-1 molecules and a C-terminal cytoplasmic end which is able to bind microtubules (or tubulin), which has the effect of anchoring the gp19k protein in the RE membrane. Gp19k is thus able to prevent expression of the MHC-1 molecules at the surface of the cells by interacting with the molecules and sequestering them within the RE. However, protein gp19k is weakly expressed by adenoviruses in the absence of viral replication. Furthermore, expression of gp19k is also dependent on a splicing taking place. Introduction of a recombinant DNA which contains a (preferably cDNA) sequence encoding gp19k into the vectors of the invention enables the expression of said protein to be controlled and optimized. In particular, the use of constitutive promoters and suppression of the other reading frames enables expression of this protein to be strongly increased and freedom to be achieved from dependence on viral replication and the presence of inducing elements. This makes it possible, particularly advantageously, to considerably diminish lysie of the infected cells by the CTL and thus to increase and prolong the in vivo production of the therapeutic gene.

Other proteins encoded by the E3 region of the adenovirus genome, such as the 10.4k and 14.5k proteins, exhibit certain properties which are attractive with regard to incorporating these genes into the vectors of the invention.

The ICP47 gene of herpes simplex virus represents another immunoprotective gene which in particularly attractive within the meaning of the present invention. Cells which are infected by herpes simplex virus exhibit resistance to lysis induced by CTLs. It has been demonstrated that the ICP47 gene, which can reduce expression of MHC-I molecules at the surface of cells, was able to confer this resistance. Incorporation of the ICP47 gene into a recombinant DNA according to the invention also enables the recombinant viruses of the invention to evade the mane system.

The UL18 gene of cytomegalovirus represents another preferred example of an immunoprotective gene according to the invention. The product of the UL18 gene is able to bind β2-microglobulin (Brown et al. Nature 347 (1990) 770). β2-Microglobulin is one of the chains of MHC-I molecules. Incorporation of the ULlB gene into a recombinant DNA according to the invention thus makes it possible to decrease the number of functional β2-microglobulin molecules in cells infected by the viruses of the invention and therefore to decrease the ability of these cells to produce MHC-I molecules which are complete and functional. This type of construct therefore enables the infected cells to be protected from lysis by CTLs.

As indicated above, the immunoprotective gone which is used within the scope of the present invention is, in another preferred embodiment, a gene whose product inhibits the activity or the signalling pathways of cytokines. The cytokines represent a family of secreted proteins which act as signal molecules for the immune system. They can attract cells of the immune system, activate them and induce them to proliferate, and can even act directly on the infected cells in order to kill them.

Among the genes whose product affects the activity or the signalling pathways of the cytokines, mention may be made of the genes which are involved in the synthesis of the cytokines or whose product is able to sequester cytokines, antagonize their activity or interfere with the intercellular signalling pathways. Preferred examples which may be cited are, in particular, the BCRF1 gene of Epstein Barr virus, the crmA and crmB genes of cowpox virus, the B15R and B18R genes of vaccinia virus, the US28 gene of cytomegalovirus, and the E3-14.7, E3-10.4 and E3-14.5 genes of adenovirus.

The B15R gone of vaccinia virus encodes a soluble protein which is able to bind interleukin-1β (the secreted form of interleukin-1) and thereby prevent this cytokine from binding to its cellular receptors. Thus, interleukin-1 is one of the first cytokines to be produced in response to an antigenic attack and it plays a very important role in the signalling of the immune system at the beginning of the infection. The feasibility of incorporating the B15R gene into a vector according to co the invention advantageously makes it possible to reduce the activity of IL-1β, in particular on the activation of the immune cells, and, therefore, provide local protection of the cells which are infected with the viruses of the invention from a significant immune response. Genes which are homologous to the B15R gene, such as the gene of cowpox virus, can also be employed.

In the same way, the B18R gone of vaccinia virus encodes a protein which is homologous to the receptor for interleukin-6. This gene, or any functional homologue, can also be used in the vectors of the invention in order to inhibit binding of interleukin-6 to its cell receptor and thus to reduce the immune response locally.

The crmB gene of cowpox virus can also advantageously be used in a similar fashion. Thus, this gone encodes a secreted protein which is able to bind TNF and to compete with the TNF receptors at cell surfaces. This gene therefore makes it possible, in the viruses of the invention, to locally decrease the concentration of active TNF which is able to destroy the infected cells. Other genes which encode proteins which are able to bind TNF and at least partially inhibit its binding to its receptors can also be employed.

The crmA gene of cowpox virus encodes a protein which has a protease-inhibiting activity of the serpin type and which is able to inhibit the synthesis of interleukin-1β. This gene can therefore be used in order locally to decrease the concentration of interleukin-1 and thus to reduce development of the immune and inflammatory responses.

The BCRF1 gene of Epstein Barr virus encodes an analogue of interleukin 10. The product of this gene is a cytokine which is able to decrease the immune response and to alter its specificity while inducing proliferation of B lymphocytes.

The US28 gene of cytomegalovirus encodes a protein which is homologous to the receptor for macrophage inflammatory protein 1α (MIP-1α). This protein is therefore able to compete with the receptors for MIP and therefore to inhibit its activity locally.

The product of the E3-147, E3-10.4 and E3-14.5 genes of adenovirus is able to block transmission of the intercellular signal which is mediated by certain cytokines. When the cytokines bind to their receptor at the surface of an infected cell, a signal is transmitted to the nucleus in order to induce cell death or stop protein synthesis. This is particularly the case for tumour necrosis factor (TNP). Incorporation of the E3-14.7, E3-10.4 and/or 93-14.5 genes into a recombinant DNA according to the invention for the purpose of expressing them constitutively or in a regulated manner enables intercell signalling which is induced by TNF to be blocked and thus cells which are infected with the recombinant viruses according to the invention to be protected from the toxic effects of this cytokine.

A local and transitory inhibition can be particularly advantageous. This can be obtained, in particular, by the choice of specific expression signals (cytokine-dependent promoters, for example) as indicated below.

It will be understood that other genes which are homologous or which have similar functional properties can be used to construct the vectors of the invention. These different genes can be obtained by any technique which is known to the person skilled in the art and, in particular, by screening libraries, by chemical synthesis or else by mixed methods including chemical or enzymic modification of sequences obtained by screening libraries. Furthermore, these different genes can be employed alone or in combination(s).

Insertion of the genes under consideration in the form of recombinant DNAs according to the invention provides greater flexibility in the construction of the adenoviruses and enable expression of said genes to be controlled more effectively.

Thus, the recombinant DNAs (and therefore the two genes of interest) which are incorporated into the adenoviral vectors according to the present invention can be organized in different ways.

They can, first of all, be inserted into the same site in the adenovirus genome or into different, selected sites. In particular, the recombinant DNAs can be inserted, at least in part, into the E1, E3 and/or E4 regions of the adenovirus genome to replace or supplement viral sequences.

Preferably, the recombinant DHAs are inserted, at least in part, within the E1, E3 or 34 regions of the adenovirus genome. When they are inserted into two different sites, preference is given, within the scope of the invention, to using the E1 and E3 regions or E1 and E4 regions. Thus, as the examples demonstrate, this organization enables the two genes to be expressed at an elevated level without interfering with each other. Advantageously, the recombinant DNAs are inserted in place of viral sequences.

These recombinant DHAs can then each include a transcriptional promoter which in identical or different. This configuration enables higher levels of expression to be achieved and provides improved control of the expression of the genes. In this case, the two genes can be inserted in the same orientation or in opposite orientations.

They can also constitute a single transcriptional entity. In this configuration, the two recombinant DNAs are contiguous and positioned such that the two genes are under the control of a single promoter and give rise to a single premessenger RNA. This arrangement is advantageous since it enables a single transcriptional promoter to be used.

Finally, the use of recombinant DRAs according to the invention makes it possible to employ transcriptional promoters of different types and, in particular, promoters which are strong or weak, regulated or constitutive, tissue-specific or ubiquitous, etc.

The choice of the expression signals and the respective positions of the DNA recombinants is particularly important as regards obtaining an elevated expression of the therapeutic gene and a significant immunoprotective effect.

A particularly preferred embodiment of the present invention employs a defective adenovirus which includes a first recombinant DNA, containing a therapeutic gene, and a second recombinant DNA, containing an immunoprotective gone, in which virus the two recombinant DNAs are inserted within the E1 region.

A particularly preferred embodiment of the present invention employs a defective adenovirus which includes a first recombinant DNA, which contains a therapeutic gene and which is inserted within the E1 region, and a second recombinant DNA, which contains an immunoprotective gene and which is inserted within the E3 region.

As indicated above, the adenovirus of the present invention are defective, that is they are unable to replicate autonomously in the target cell. Generally, the genome of the defective adenoviruses according to the present invention therefore lacks at least the sequences which are required for replicating said virus in the infected cell. These regions can be eliminated (in whole or in part), rendered non-functional, or substituted by other sequences and, in particular, by the therapeutic genes. The defective character of the adenoviruses of the invention is an important feature since it ensures that the vectors of the invention are not disseminated following administration.

In a preferred embodiment, the adenoviruses of the invention encompass ITR sequences and an encapsidation sequence, and possess a deletion of all or part of the E1 gene.

The inverted repeat (ITR) sequences represent the origin of replication of the adenovirus. They are located at the 3′ and 5′ ends of the viral genome (cf. FIG. 1), from where they can easily be isolated using standard molecular biological techniques known to the person skilled in the art. The nucleotide sequence of the ITR sequences of the human adenoviruses (in particular serotypes Ad2 and Ad5) is described in the literature, as are those of the canine adenoviruses (in particular CAV1 and CAV2). In the case of the Ad5 adenovirus, for example, the left-hand ITR sequence corresponds to the region encompassing nucleotide 1 to 103 of the genome.

The encapsidation sequence (also termed Psi sequence) is required for encapsidating the viral DNA. This region must, therefore, be present to enable defective recombinant adenoviruses according to the invention to be prepared. In the adenovirus genome, the encapsidation sequence is located between the left-hand (5′) ITR and the E1 gene (cf. FIG. 1). It can either be isolated or synthesized artificially using standard molecular biological techniques. The nucleotide sequence of the encapsidation sequence of human adenoviruses (in particular serotypes Ad2 and Ad5) is described in the literature, as are those of the canine adenoviruses (in particular CAV1 and CAV2). In the case of the Ad5 adenovirus, for example, the encapsidation sequence corresponds to the region encompassing nucleotides 194 to 358 of the genome.

More preferably, the adenoviruses of the invention encompass the ITR sequences and an encapsidation sequence, and possess a deletion of all or part of the E1 and E4 genes.

In a particularly preferred embodiment, all or part of the E1, E3 and E4 genes and, even more preferably, all or part of the E1, E3, L5 and E4 genes are deleted from the genome of the adenoviruses according to the invention.

The adenoviruses of the invention can be prepared from adenoviruses of varying origin. Thus, different serotypes of adenovirus exist whose structures and properties vary to some extent but which exhibit a comparable genetic organization. Consequently, the teaching described in the present application can easily be reproduced by the person skilled in the art for any type of adenovirus.

More specifically, the adenoviruses of the invention can be of human, animal or mixed (human and animal) origin.

As regards adenoviruses of human origin, preference is given to using those which are classed within the C group. More preferably, preference is given, among the different serotypes of human adenovirus, to using, within the scope of the present invention, type 2 or type 5 (Ad 2 or Ad 5) adenoviruses.

As indicated above, the adenoviruses of the invention can also be of animal origin or include sequences which are derived from adenoviruses of animal origin. Thus, the Applicant has demonstrated that adenoviruses of animal origin are able to infect human cells in a highly efficient mannor and that they are unable to propagate themselves in the human cells in which they have been tested (cf. Application FR 93 05954). The Applicant has also demonstrated that the adenoviruses of animal origin are in no way trans-complemented by adenoviruses of human origin, thereby eliminating any risk of recombination and propagation in vivo in the presence of a human adenovirus, which may lead to formation of an infectious particle. The use of adenoviruses or of adenovirus regions of animal origin is therefore particularly advantageous since the risks which are inherent in the use of viruses as vectors in gene therapy are even lower.

The adenoviruses of animal origin which can be used within the scope of the present invention can be of canine, bovine, murine (example: Mav 1, Beard et al., Virology 75 (1990) 81), bovine, porcine, avian or else simian (example: SAV) origin. More specifically, those avian adenoviruses which may be mentioned are serotypes 1 to 10 which are available from the ATCC, such as, for example, the Phelps (ATCC VR-432), Fonteo (ATCC VR-280), P7-A (ATCC VR-827), IBH-2A (ATCC VR-828), J2-A (ATCC VR-829), TS-A(ATCC VQ-830) or K-11 (ATCC VR-921) strains or else the strains referenced ATCC VR-831 to 835. Those bovine adenoviruses which may be used are the different known serotypes, in particular those which are available from the ATCC (types 1 to 8) under reference numbers ATCC VR-313, 314, 639-642, 768 and 769. Murine adenoviruses FL (ATCC VR-550) and E20308 (ATCC VR-528), ovine adenovirus type 5 (ATCC VR-1343) or type 6 (ATCC VR-1340), porcine adenovirus 5359, or simian adenoviruses such as, in particular, the adenoviruses referenced at ATCC under numbers VR-591-594, 941-943, 195-203, etc., may also be mentioned.

Among the different adenoviruses of animal origin, preference is given, within the scope of the invention, to using adenoviruses or adenovirus regions of canine origin, in particular all the strains of the CAV2 [Manhattan strain or A26/61 strain (ATCC VR-800), for example] adenoviruses. The canine adenoviruses have been the subject of numerous structural studies. Thus, complete restriction maps of adenoviruses CAV1 and CAV2 have been described in the prior art (Spibey et al., J. Gen. Virol 70 (1989) 165), and the Bla and 83 genes as well as the ITR sequences have been cloned and sequenced (see, in particular, Spibey et al., Virus Res. 14 (1989) 241; Linné, Virus Res. 23 (1992) 119, WO 91/11525). The defective recombinant adenovirus according to the invention can be prepared in different ways.

A first method consists in transfecting the DNA of the defective recombinant virus, which has been prepared in vitro (either by ligation or in plasmid form), into a competent cell line, that is a cell line which carries, in trans, all the functions which are required for complementing a defective virus. These functions are preferably integrated into the genome of the cell, thereby enabling the risks of recombination to be avoided and conferring increased stability on the cell line.

A second approach consists in co-transfecting the DNA of the defective recombinant virus, which has been prepared in vitro (either by ligation or in plasmid form), and the DNA of a helper virus into an appropriate cell line. When this method is used, it is not necessary to have available a competent cell line which is able to complement all the defective functions of the recombinant adenovirus. This is because some of these functions are complemented by the helper virus. This helper virus should itself be defective, and the cell line then carries in trans the functions which are required for complementing it. Of the cell lines which can be used, in particular, within the scope of this second approach, those which may be mentioned, in particular, are the human embryonic kidney line 293, KB calls, Hela, MDCK and GEK cells, etc. (cf. examples).

Subsequently, the vectors which have multiplied are recovered, purified and amplified using standard molecular biological techniques.

According to one embodiment, it is possible to prepare the DNA of the defective recombinant virus carrying the appropriate deletions and the two recombinant DNAs in vitro, either by ligation or in plasmid form. As indicated above, the vectors of the invention advantageously possess a deletion of all or part of certain viral genes, in particular the E1, E3, E4 and/or L5 genes. This deletion can correspond to any type of suppression which affects the gene under consideration. It can, in particular, be a question of deletion of all or part of the coding region of said gene and/or all or part of the promoter region for transcribing said gene. The deletion is generally carried out on the DNA of the defective recombinant virus by, for example, digesting with appropriate restriction enzymes and then ligating, using molecular biological techniques as illustrated in the examples. The recombinant DNAs can then be inserted into this DNA, by enzymic cleavage followed by ligation, within selected regions and in the chosen orientation.

The DNA which is thus obtained, and which consequently carries the appropriate deletions and the two recombinant DNAs, enables the defective recombinant adenovirus, carrying the said deletions and recombinant DNAs, to be generated directly. This first variant is particularly well suited for achieving recombinant adenoviruses in which the genes are arranged in the form of a single transcriptional unit, or under the control of separate promoters but inserted into the same site in the genome.

It is also possible to prepare the recombinant virus in two steps, enabling the two recombinant DNAs to be introduced successively. In this case, the DNA of a first recombinant virus, carrying the appropriate deletions (or some of said deletions), and one of the recombinant DNAs is constructed, by ligation or in plasmid form. This DNA is then used to generate a first recombinant virus which carries said deletions and one recombinant DNA. The DNA of this first virus is then isolated and co-transfected with a second plasmid or the DNA of a second defective recombinant virus which carries the second recombinant DNA, the appropriate deletions (that part not present on the first virus) and a region permitting homologous recombination. This second step thereby generates the defective recombinant virus carrying the two recombinant DNAs. This preparation variant is particularly suitable for preparing to recombinant viruses which carry two recombinant DNAs which are inserted into two different regions of the genome of the adenovirus.

The two agents according to the invention, namely the immunosuppressant and the recombinant adenovirus, can be formulated with a view to administering them by any of the topical, oral, parental, intranasal, intravenous, intramuscular, subcutaneous, intraocular, tranudermal, atc. routes.

Preferably, the respective pharmaceutical formulation(s) contain(s) excipient which are pharmaceutically acceptable for an injectable formulation. These excipients can, in particular, be sterile, isotonic salt solutions (monosodium or disodium phosphate, sodium, potassium, calcium or magnesium chloride, etc., or mixtures of such salts), or dry, in particular lyophilized, compositions which, by adding, as the case may be, sterilized water or physiological saline, enable injectable solutions to be constituted.

The doses of immunosuppressant and of adenovirus which are used for the injection can be adapted in accordance with different parameters, in particular in accordance with the mode of administration which is used, the pathology concerned, the gene to be expressed, or else the sought-after duration of the treatment.

In a general manner, the recombinant adenoviruses according to the invention are formulated and administered in the form of doses containing between 10⁴ and 10¹⁴ pfu/ml, preferably from 10⁶ to 10¹⁰ pfu/ml. The term pfu (“plaque-forming unit”) corresponds to the infective power of a solution under consideration and is determined by infecting a suitable cell culture and measuring, generally after 5 days, the number of plaques of infected cells. The techniques for determining the pfu titre of a viral solution are well documented in the literature. As far as the immunosuppressants, more specifically, are concerned, their doses and modes of injection vary in accordance with their nature. Adjustment of these two parameters comes within the competence of the person skilled in the art.

The medicinal combination according to the invention can be used for treating or preventing numerous pathologies. Depending on the therapeutic gene which is inserted into its adenovirus, it can be used, in particular, for treating or preventing genetic disorders (dystrophy, mucoviscidosis, etc.), neurodegenerative diseases (Alzheimer's, Parkinson's, ALS, etc.), hyperproliferative pathologies (cancers, restenosis, etc.), pathologies associated with coagulation disorders or with dyslipoproteinamias, pathologies associated with viral infections (hepatitis, AIDS, etc.), etc.

The present invention also relates to any method of therapeutic treatment which employs the claimed medicinal combination.

The present invention will be more completely described with the aid of the examples which follow and which should be considered as being illustrative and not limiting.

FIG. 1: Genetic organization of the Ads adenovirus. The complete sequence of Ad5 is available on database and enables the person skilled in the art to select or create any restriction site and thus to isolate any region of the genome.

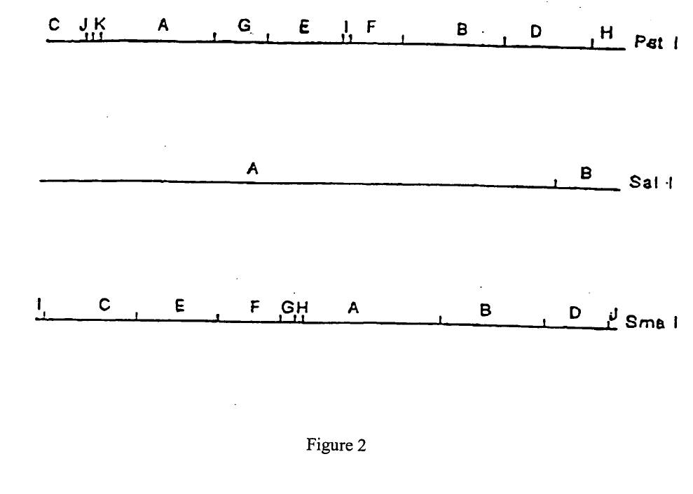

FIG. 2: Restriction map of the Manhattan strain of the CAV2 adenovirus (according to Spibey at al. cited above)

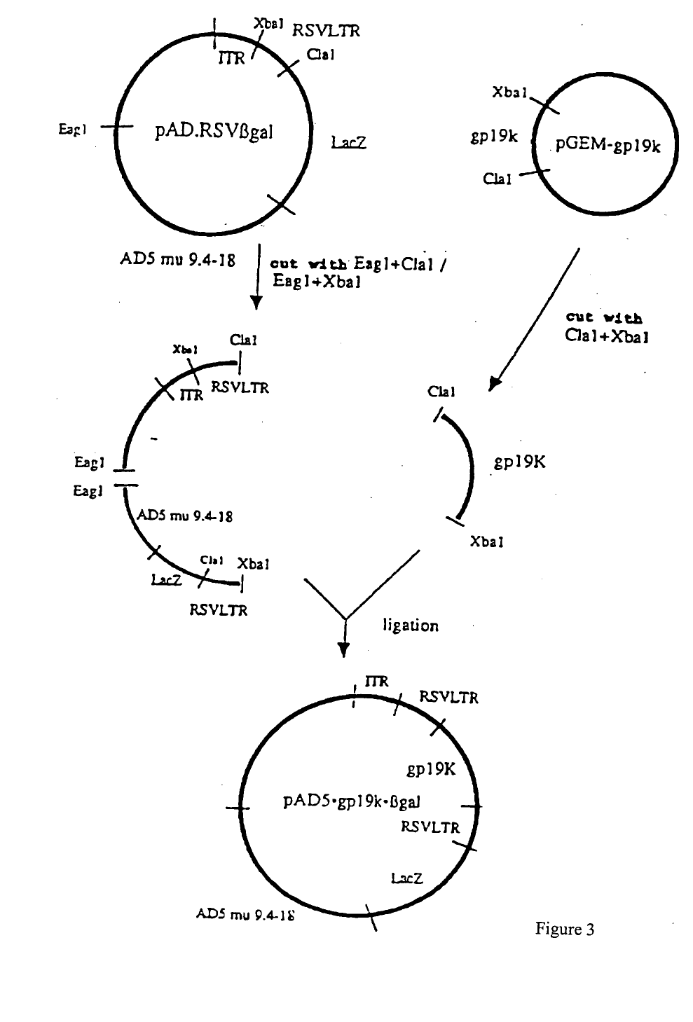

FIG. 3: Construction of the vector pAD5-gp19k-βgal.

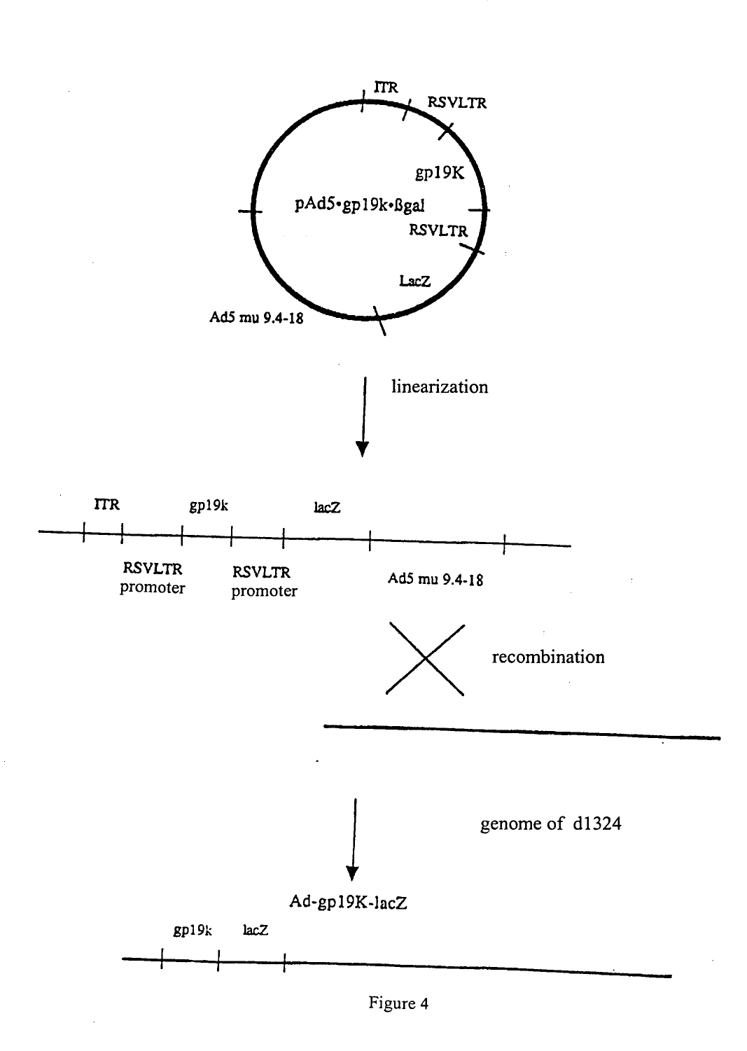

FIG. 4: Construction of the adenovirus Ad-gp19k-βgal, ΔE1, ΔE3.

### General Molecular Biological Techniques

The methods which are routinely used in molecular biology, such as preparative extractions of plasmid DNA, centrifugation of plasmid DNA in a caesium chloride gradient, electrophoresis on agarose or acrylamide gels, purification of DNA fragments by electroelution, extraction of proteins with phenol or with phenol/chloroform, precipitation of DNA in a saline medium with ethanol or with isopropanol, transformation into Bscherichia coli, etc. are well known to the person skilled in the art and are amply described in the literature [Maniatis T. at al., “Molecular Cloning, A Laboratory Manual”, Cold Spring Harbor Laboratory, Cold Spring Harbor, N.Y., 1982; Ausubel F.M. et al. (ads), “Current Protocols in Molecular Biology”, John Wiley & Sons, New York, 1987].

The plasmids of the pBR322 and pUC type, and the phages of the M13 series, were obtained commercially (Bethesda Research Laboratories).

For the ligations, the DNA fragments can be separated according to their size by electrophoresis in agarose or acrylamide gals, extracted with phenol or with a phenol/chloroform mixture, precipitated with ethanol and then incubated in the presence of phage T4 DNA ligase (Biolabs) in accordance with the supplier's recommendations.

The protruding 5′ ends can be filled in using the Klenow fragment of E. coli DNA polymerase I (Biolabs) in accordance with the supplier's specifications. The protruding 3′ ends are destroyed in the presence of phage T4 DNA polymerase (Biolabs) which is used in accordance with the maufacturer's recommendations. The protruding 5′ ends are destroyed by carefully treating with S1 nuclease.

In vitro site-directed mutagenesis using synthetic oligodeoxynucleotides can be carried out using the method developed by Taylor at al. [Nucleic Acids Res. 13 (1985) 8749-8764] and employing the kit distributed by Amersham.

Enzymic amplification of DNA fragments by means of the technique termed PCR [polymerase-catalyzed chain reaction, Saiki R. K. et al., Science 230 (1985) 1350-1354, Mullin K. B. and Faloona P. A., Meth. Enzym. 155 (1967) 335-350] can be carried out using a DNA thermal cycler (Perkin Elmer Cetus) in accordance with the manufacturer's specifications.

The nucleotide sequences can be verified by means of the method developed by Sanger et al. [Proc. Natl. Acad. Sci. USA, 74 (1977) 5463-5467] using the kit distributed by Amersham.

### Cell Lines Employed

In the examples which follow, the following cell lines have been or can be employed:

Human embryonic kidney line 293 (Graham et al., J. Gen. Virol. 36 (1977) 59). This line contains in particular, integrated into its genome, the left-hand part of the genome of the hi-an adenovirus AdS (12%).

KB human cell line. Derived from a human epidermal carcinoma, this line can be obtained from ATCC (ref. CCL17) as can the conditions for culturing it.

Hela human cell line: derived from a human epithelium carcinoma, this line can be obtained from ATCC (ref. CCL2) as can the conditions for culturing it.

MDCK canine call lines the conditions for culturing MDCK cells have been described, in particular, by Macatney et al., Science 44 (1988) 9.

gm DBP6 call line (Brough et al., Virology 190 (1992) 624). This line consists of Hela calls carrying the adenovirus E2 gone under the control of the LTR of MMTV.

### EXAMPLES

### Example 1

Construction of defective recombinant adeno-viruses encompassing a therapeutic gene (the LacZ gene of E. coli) under the control of the LTR promoter of RSV and the gp19k gene under the control of the LTR promoter of RSV, with both genes being inserted within the E1 region.

These adenoviruses were constructed by homologous recombination between a plasmid carrying the left-hand part of the Ad5 adenovirus, the two recombinant DNAs and a region of the Ad5 adenovirus (corresponding to protein IX) and the DNA of a defective adenovirus carrying various deletions.

1. Construction of the Vector pAD5-gp19k-βgal (FIG. 3)

1.1. Construction of the plasmid pGEH-gp19k

Plasmid pAD5-gp19k-βgal contains a cDNA sequence encoding the adenovirus protein gp19k. This plasmid was constructed as follows. The XbaI fragment of the genome of wild-type Ad5 adenovirus, containing the E3 region, was isolated and cloned into the corresponding site of plasmid pGEM (Promega) in order to generate plasmid pGEM-23. The HinfI fragment, containing the sequence encoding gp19k (nucleotide 28628 to 29634 of wild-type Ad5 adenovirus), was then isolated from plasmid pGEM-E3. The end of this fragment were rendered blunt by the action of the Klenow fragment of E. coli DNA polymerase I (cf. general molecular biological techniques) and the fragment which was obtained was then cloned into the Smal site of plasmid pGEMzf+ (Promega).

The plasmid which was obtained was designated pGEM-gp19k (FIG. 3).

1.2. Construction of the vector pAD5-gp19k-βgal

This rumple describes the construction of a plasmid which contains one of the two recombinant DNAs encompassing their own promoter, the left-hand part of the adenovirus genome and a supplementary part (protein pIX) permitting homologous recombination. This vector was constructed from the plasmid pAd.RSVβGal as follows.

The plasmid pAd.RSVβGal contains, in the 5′>3′ orientation,

the PvuII fragment corresponding to the left-hand end of adenovirus Ad5 encompassing: the ITR sequence, the origin of replication, the encapsidation signals and the E1A enhancer,

the gene encoding β-galactosidase under the control of the RSV promoter (from Rous sarcoma virus).

a second fragment of the genome of adenovirus Ad5, which permits homologous recombination between plasmid pAd.RSVβGal and the adenovirus d1324. Plasmid pAd.RSVβGal has been described by Stratford-Perricaudet et al. (J. Clin. Invest. 90 (1992) 626).

Plasmid pAd.RSVβGal was first of all cut with the enzymes BagI and Clal. This generates a first fragment carrying, in particular, the left-hand part of adenovirus Ad5 and the LTR promoter from RSV. In parallel, the plasmid pAd.RSVβGaI was also cut with the enzymes BagI and XbaI. This generates a second type of fragment carrying, in particular, the LTR promoter of RSV, the LacZ gene and a fragment of the genome of adenovirus Ad5 which permits homologous recombination. The ClaI-EagI and EagI-XbaI fragments were then ligated in the presence of the XbaI-ClaI fragment from plasmid pGEM-gp19k (Example 1.1) carrying the sequence encoding gp19k (cf. FIG. 3). The vector which was obtained in this way, designated pAD5-gp19k-βgal, therefore contains

the PvuII fragment corresponding to the left-hand end of adenovirus Ad5 encompassing: the ITR sequence, the origin of replication, the encapsidation signals and the E1A enhancer,

the sequence encoding gp19k under the control of the RSV promoter (from Rous sarcoma virus),

the gene encoding β-galactosidase under the control of the RSV promoter (from Rous sarcoma virus), and

a second fragment of the genome of adenovirus Ad5 which permits homologous recombination.

2. Construction of the recombinant adenoviruses

2.1. Construction of a Recombinant Adenovirus which is deleted in the E1 region and which carries the two recombinant DNAs inserted in the same orientation within the E1 region.

Vector pAD5-gp19k-βgal was linearized and cotransfected with an adenoviral vector, which was deficient in the E1 gene, into helper cells (line 293) which supplied in trans the functions encoded by the adenovirus E1 (E1A and E1B) regions.

More precisely, the adenovirus Ad-gp19k-βgal, ΔE1 is obtained by homologous recombination in vivo between the adenovirus Ad-RSVβgal (cf. Stratford-Perricaudet et al. cited above) and vector pAD5-gp19k-βgal in accordance with the following protocol: plasmid pAD5-gp19k-βgal, which is linearized with XmnI, and adenovirus Ad-RSVβgal, which is linearized with the enzyme ClaI, are co-transfected into line 293 in the presence of calcium phosphate in order to enable homologous recombination to take place. The recombinant adenoviruses which are generated in this way are then selected by plaque purification. Following isolation, the DNA of the recombinant adenovirus is amplified in cell line 293, resulting in a culture supernatant which contains the unpurified defective recombinant adenovirus with a titre of approximately 10¹⁰ pfu/ml.

In general, the viral particles are purified by centrifugation in a caesium chloride gradient in accordance with known techniques (see, in particular, Graham et al., Virology 52 (1973) 456). The adenovirus w Ad-gp19βgal,AE1 can be stored at −80° C. in 20% glycerol.

2.2 Construction of a recombinant adenovirus which is deleted in the E1 and E3 regions and which carries the two recombinant DNAs inserted in the same orientation within the E1 region (FIG. 4).

Vector pAD5-gp19k-βgal was linearized and cotransfected with an adenoviral vector, which was deficient in the E1 and E3 genes, into helper cells (line 293) which supply in trans the functions encoded by the adenovirus E1 (E1A and E1B) regions.

More precisely, the adenovirus Ad-gp19k-βgal, ΔE1, ΔE 3 was obtained by homologous recombination in vivo between the mutant adenovirus Ad-d11324 (Thimmappaya et al, Cell 31 (1982) 543) and vector pAD5-gp19k-βgal in accordance with the following protocol: plasmid pAD5-gp 19k-βgal and adenovirus Ad-d11324, linearized with the enzyme ClaI, were cotransfected into line 293 in the presence of calcium phosphate in order to enable homologous recombination to take place. The recombinant adenoviruses which were generated in this way were then selected by plaque purification. Following isolation, the DNA of the recombinant adenovirus is amplified in cell line 293, resulting in a culture supernatant which contains the unpurified defective recombinant adenovirus with a titre of approximately 10¹⁰ pfu/ml.

In general, the viral particles are purified by centrifugation in a caesium chloride gradient in accordance with known techniques (see, in particular, Graham et al. Virology 52 (1973) 456). The genome of the recombinant adenovirus was then verified by Southern blot analysis. Adenovirus Ad-gp19k-βgal, ΔE1, ΔE3 can be stored at −80° C. in 20% glycerol.

### Example 2

Demonstration of the immunoprotective activity of the medicinal combination according to the invention.

60 adult female DBA/2 mice are divided randomly into 6 groups of 10 mice which are then treated respectively in accordance with the following injection protocols:

GROUP 1a:

Is given an intraocular injection of 10 μg of anti-CD3 monoclonal antibodies on days −2, −1, 1, 2, 3, 4 and 5 with an intravenous injection of 4.10⁹ pfu of Ad-RSVβgal virus on day 0 (cf. Stratford-Perricaudet et al. cited above).

GROUP 1b:

Is given the same treatment as group 1a but employing, as virus, 4.10⁹ pfu of Ad-gp 19k-βgal virus (FIG. 4).

GROUP 2a:

Is given an intraperitoneal injection of 250 μg of anti-CD4 monoclonal antibodies on days −2, −1, 1, 4 and 7 with an intravenous injection of 4.10⁹ pfu of Ad-RSV βgal virus on day 0.

GROUP 2b:

Is given the same treatment as group 2a but using, as virus, 4.10⁹ pfu of Ad gp 19k-βg al virus.

GROUP 3a:

Is given an intravenous injection of 4.10⁹ pfu of Ad-βgal without any accompanying administration of immunosuppressant.

GROUP 3b:

Is given an intravenous injection of 4.10⁹ pfu of Ad-gp19k-βgal without any accompanying administration of immunosuppressant.

At various times, two animals from each group were sacrificed with the aim of removing their livers and spleens.

2.1 —Immunofluorescence analysis of the distribution of the principal lymphocyte subpopulations (CD3+, CD4+ and CD8+) within oplenocytes which, are removed on D15 after the injection.

A suspension of isolated cells was prepared from removed spleens. A cell sample was analysed by immunofluorescence using antibodies which were specific for each lymphocyte subpopulation. The fluorescent cells ere read with the aid of a cytofluorimeter (Becton Dickinson FACS Scan). The results are given in Table I below.

TABLE I

| | | | Group 3b | | Group 1a | | Group 2a | |
|---|---|---|---|---|---|---|---|---|
| | Group 3a | | Ad-βgal- | | anti-CD3/ | | anti-CD4/ | |
| | Ad-βgal | | gp19k | | Ad-βgal | | Ad/βgal | |
| | % of cells expressing βgal | | | | | | | |
| | at the cell surface | | | | | | | |
| CD3 | 20.0 | 17.5 | 20.6 | 21 | 5.4 | 6.1 | 12 | 10.3 |
| CD4 | 13.4 | 12.6 | 15.3 | 16.8 | 4.4 | 5.1 | 2.7 | 4.1 |
| CD8 | 5.5 | 5.5 | 6.1 | 6 | 2.02 | 2.3 | 7.9 | 6.6 |

The clear decrease in the CD3+, CD4+ and CDS+ 10 cells in the animals treated with anti-CD3 is noted as is the selective decrease in CD4+cells in the animals treated with anti-CD4.

2.2.—Analysis of the cytotoxic capacity of the splenocytes which are removed at D32 after the injection and stimulated in vitro with regard to histocompatible target cells expressing βgal

A second splenocyte sample isolated from the spleens of treated animals was cultured in vitro for 4 days in the presence of P815 cells expressing β-galactosidase at their surface. At the end of the culture, the cytotoxic activity of these splenocytes with w regard to P815-βgal target cells labelled with Cr⁵¹ was evaluated. The cytotoxic activity, expressed as per cent cytolysis, was determined in a conventional manner by bringing together different ratios of effector cells and target cells. The results are presented in Table II below.

TABLE II

| | Group | |
|---|---|---|
| | 2b | |
| | Anti- | |
| | CD4/ | |
| | Ad- | 3A |
| | βgal- | AD- |
| Treatment | gp19k | βgal |
| Ratio | | |

| | |
|---|---|
| Effector/targets | % cytolysis |

| | | | | |
|---|---|---|---|---|
| 80/1 | 4 | 2 | 13 | 14 |
| 40/1 | 2 | 1 | 13 | 9 |
| 20/1 | 1 | 1 | 5 | 9 |
| 10/1 | 0 | 0 | 2 | 5 |
| 5/1 | 0 | 1 | 1 | 2 |

There is seen to be a very clear neutralization of the cytotoxic capacity of the splenocytes which were removed from the animals having been treated with anti-CD4, that is to say the group 2b.

2.3. Expression of β-galactosidase activity in the liver after 15 and 32 days.

The livers are sectioned and stained with X-gal in order to display the β-galactosidase activity and with resin in order to demonstrate the histology of the section. The results are presented in Table III below.

TABLE III

Number of cells
expressing βgal

| | 15 days | 32 days |
|---|---|---|
| Group 2a: | 1 | 1 |
| (anti-CD4/Ad-βgal) | | |
| Group 2b: (anti-CD4/Ad | 250 | 50 |
| gp19k-βgal) | | |
| Group 3a: (Ad-βgal) | 3 | 0 |
| Group 3b: | 25 | 0 |
| (Ad gp19k-βgal) | | |

From the results presented above, it emerges that injection of anti-CD4 antibodies in association with an injection of Adgp19k-βgal induces an expression of the gene under consideration which is markedly prolonged. Thus, 30 days after the injections, significant β-galactosidase activity is observed in the case of group 2b. This prolongation, which can be interpreted as the result of a tolerance phenomenon which is induced in accordance with the invention, is markedly greater than that which could have been expected from the simple juxtaposition of the respective effects of the anti-CD4 immunosuppressants and of the recombinant adenovirus Ad gp19k-βgal.

Furthermore, no inflammatory reaction is observed over this period of 30 days in the case of group 2b.

1. A composition comprising an immunosuppressive agent and a recombinant adenovirus, wherein the genome of the adenovirus comprises a first recombinant DNA and a second recombinant DNA, wherein the second recombinant DNA contains a sequence coding for an adenoviral gp 19k protein.

2. The composition according to claim 1, wherein the immunosuppressive agent is selected from cyclosporin, FK506, azathioprine, a coiticosteroid, or a monoclonal antibody or polyclonial antibody that is able to inactivate an immune molecule or induce destruction of an immune cell carrying these molecules.

3. The composition according to claim 2, wherein the antibody is selected from the group anti-CD4, -CD2, -CD3, -CD8, -CD28, -B7, -ICAM-1 and -LFA-1 antibodies, and CTLA4Ig.

4. The composition according to claim 1, wherein the first recombinant DNA encodes a protein.

5. The composition according to claim 1, wherein the first recombinant DNA encodes a human protein.

6. The composition according to claim 1, wherein the first recombinant DNA encodes a ribozyme or anitisenise RNA.

7. The composition according to claim 1, wherein the first and second recombinant DNAs constitute a single transcriptional entity.

8. The composition according to claim 1, wherein the first and second recombinant DNAs include an identical transcriptional promoter.

9. The composition according to claim 8, wherein the first and second recombinant DNAs are inserted in the same orientation.

10. The composition according to claim 1, wherein the first and second recombinant DNAs are inserted into the same region of the adenovirus genome.

11. The composition according to claim 10, wherein the first and second recombinant DNAs are inserted within the E1, E3, or E4 regions.

12. The composition according to claim 1, wherein the first and second recombinant DNAs are inserted into different sites in the adenovirus genome.

13. The composition according to claim 12, wherein one of the first or second recombinant DNAs is inserted within the E1 region and the other within the E3 or E4 region.

14. The composition according to claim 1, wherein the adenovirus is a defective recombinant adenovirus, which encompasses the ITR sequences and a sequence permitting encapsidation, and which carries a deletion of all or part of the E1 and E4 genes.

15. The composition according to claim 1, wherein the adenovirus contains a deletion in all or part of the E1, E3, L5 and E4 genes.

16. The composition according to claim 1, wherein the recombinant adenovirus genome comprises a region of a human Ad 5 or Ad 2 adenovirus.

17. The composition according to claim 1, wherein the sequence coding for an adenoviral gp 19k protein is from a wild type human Ad 5 adenovirus.

18. The composition according to claim 1, wherein the sequence coding for an adenoviral gp 19k protein contains one or more point mutations compared to the wild type human Ad 5 adenovirus sequence, and wherein the gp19k protein retains an immunosuppressive activity.

19. A method for expressing a sequence of interest from an adenovirus comprising consecutively or simultaneously administering to a subject an immunosuppressive agent and a recombinant adenovirus, wherein the genome of the adenovirus comprises a first recombinant DNA comprising the sequence of interest and a second recombinant DNA containing a sequence coding for an adenoviral gp19k protein, where the sequence of interest is expressed from the adenovirus.

20. The method according to claim 19, wherein the recombinant adenovirus is administered in vivo.

21. The method according to claim 19, wherein the immunosuppressive agent is selected from cyclosporin, FK506, azathioprine, a coiticosteroid, or a monoclonal antibody or polyclonial antibody that is able to inactivate an immune molecule or induce destruction of all immune cell carrying these molecules.

22. The method according to claim 21, wherein the antibody is selected from the group anti-CD4,-CD2,-CD3,-CD8,-CD28,-B7,-ICAM-1 and -LFA-1 antibodies, and CTLA41g.

23. The method according to claim 19, wherein the sequence coding for an adenoviral gp19k protein is from a wild type human Ad 5 adenovirus.

24. The method according to claim 19, wherein the sequence coding for an adenoviral gp19k protein contains one or more point mutations compared to the wild type human Ad 5 adenovirus sequence, and wherein the gp19k protein retains an immunosuppressive activity.

25. The method according to claim 19, wherein the first recombinant DNA encodes a protein.

26. The method according to claim 19, wherein the first recombinant DNA encodes a human protein.

27. The method according to claim 19, wherein the first recombinant DNA encodes a ribozyme or antisense RNA.

28. The method according to claim 19, wherein the first and second recombinant DNAs constitute a single transcriptional entity.

29. The method according to claim 19, wherein the first and second recombinant DNAs each include an identical transcriptional promoter.

30. The method according to claim 29, wherein the first and second recombinant DNAs are inserted in the same orientation.

31. The method according to claim 19, wherein the immunosuppressive agent is administered both before and after administration of the adenovirus.

32. The method according to claim 19, wherein the immunosuppressive agent and the recombinant adenovirus are administered simultaneously.

33. The method according to claim 19, wherein the adenovirus is administered by injection.

34. The composition according to claim 1, wherein the first recombinant DNA comprises a coding sequence for p53, aFGF, bFGF, factor VIII, or factor IX.

35. The composition according to claim 34, wherein the first recombinant DNA comprises a coding sequence for p53.

36. The method according to claim 19, wherein the first recombinant DNA comprises a coding sequence for p53, aFGF, bFGF, factor VIII, or factor IX.

37. The method according to claim 36, wherein the first recombinant DNA comprises a coding sequence for p53.

38. A method of prolonging the survival of a cell expressing a sequence of interest, comprising
introducing a recombinant adenovirus to a cell of an animal, the genome of the adenovirus comprising a first recombinant DNA containing the sequence of interest and a second recombinant DNA containing a sequence coding for an adenoviral gp19k protein,
treating the animal with an immunosuppressive agent, and
detecting the presence of m.RNA or protein expressed from the sequence of interest, whereby the expression of the sequence of interest results in prolonged cell survival.

39. The method according to claim 38, wherein the sequence of interest encodes a protein, ribozyme, or antisenise RNA.

40. The method according to claim 38, wherein the sequence of interest encodes a human protein.

41. The method according to claim 40, wherein the sequence of interest comprises a coding sequence for p53, aFGF, bFG.F, factor VIII, or factor IX.

42. The method according to claim 41, wherein the sequence coding for an adenoviral gp19k protein is from a wild type human Ad 5 adenovirus.

43. The method according to claim 38, wherein the sequence coding for an adenoviral gp19k protein contains one or more point mutations compared to the wild type human Ad 5 adenovirus sequence, and wherein the gp19k protein retains an immunosuppressive activity.

44. The method according to claim 41, wherein the sequence of interest comprises a coding sequence for p53.
